(19) United States
(12) Patent Application Publication
(10) Pub. No.: US 2013/0068010 A1
(43) Pub. Date: Mar. 21, 2013

(54) Determination of Yarn Features

(71) Applicants: Norbert Ziganek, Gutenswil (CH); Martin Kuster, Eschenbach (CH)

(72) Inventors: Norbert Ziganek, Gutenswil (CH); Martin Kuster, Eschenbach (CH)

(73) Assignee: USTER TECHNOLOGIES AG, Uster (CH)

(21) Appl. No.: 13/695,841

(22) PCT Filed: Jun. 1, 2011
(86) PCT No.: PCT/CH2011/000131

§ 371 Date: Nov. 2, 2012

(30) Foreign Application Priority Data
Jun. 9, 2010 (CH) 924/10

(51) Int. Cl. G01N 33/36

(52) U.S. Cl. CPC G01N 33/36
USPC 73/159

(57) ABSTRACT

The device for determining features, in particular the hairiness, of a yarn includes a housing and a sensor unit attached in or on the housing for measuring at least one parameter of the yarn. The housing includes a front plate, a rear plate, and a frame located between the front plate and the rear plate. The frame is produced integrally and separately from the front plate and the rear plate, for example from a bent metal sheet. The front plate and the rear plate are designed as flat plates and fastened, by means of screw connections, to lugs that are bent inward on the frame. The housing thus has a particularly simple design.

### BACKGROUND

The present invention lies in the field of quality control in the textile laboratory. It relates to an apparatus for determining features, especially the hairiness, of a yarn, according to the preambles of the independent claims. Such apparatuses are typically used in a textile laboratory.

In the case of staple fiber yarns, which are spun of many fibers, individual fibers are not incorporated completely into the actual yarn body. They partly protrude from the yarn body, which is known as hairiness of the yarn. The hairiness is an important yarn property, which is why the textile industry is highly interested in reliably and reproducibly measuring and classifying the same.

Apparatuses for determining the hairiness of yarns are known, e.g. the device USTER® ZWEIGLE HAIRINESS TESTER 5 of the applicant, which has been described for example in “USTER® ZWEIGLE HAIRINESS TESTER 5 Application Handbook”, V1.0, September 2009, or the device USTER® TESTER 5-S800 of the applicant, which has been described for example in the brochure “USTER® TESTER 5-5800 Brilliant Testing—Superior Analysis”, August 2007. The specifications EP-0,271,728 A2, EP 1,621,872 A2, DE-198,18,069 A1 and DE 199,24,840 A1 also disclose hairiness measuring devices.

U.S. Pat. No. 3,566,194 A describes an electrical distribution board with an especially flat housing. The four housing walls are provided with inwardly bent lugs on which a front plate is fastened.

EP-0,266,612 A2 discloses a yarn testing device with two feed rollers which rest on each other for conveying the yarn to be tested. During testing operation, a bearing arm of the one feed roller presses a safety switch. As soon as the bearing arm with the feed roller is pivoted away, the safety switch switches off the drive motors of the feed rollers.

U.S. Pat. No. 4,213,056 A discloses an apparatus for testing interlaced multifilament yarns. The apparatus contains an optical sensor unit, in which a contact body is disposed. The multifilament yarn to be tested is guided with a predetermined contact pressure over the contact body and is optically scanned. A curved insertion slit is provided in the bottom and top cover plate of the sensor unit for introducing the multifilament yarn in such a way that it is guided wrapped around the contact body.

### SUMMARY

It is an object of the present invention to further improve the known apparatuses for determining features of a yarn concerning various aspects.

This and further objects are achieved by the apparatus in accordance with the invention, as defined in the independent claims. Advantageous embodiments are provided in the dependent claims.

In a first embodiment, the apparatus in accordance with the invention for determining features of a yarn contains a housing and a sensor device attached to the housing for measuring at least one parameter of the yarn. The housing substantially consists of a front plate, a rear plate and a frame disposed between the front plate and the rear plate. The frame is produced integrally and separately from the front plate and the rear plate. The frame is preferably made of a bent sheet metal. The front plate and the rear plate can be designed as flat plates and be fastened by means of screw connections to lugs which are bent inwardly on the frame. The housing has therefore an especially simple configuration in this first embodiment.

In a second embodiment, the apparatus in accordance with the invention for determining features of a yarn contains a housing with a front plate standing substantially perpendicularly on a base, a sensor device attached in or to the housing for measuring at least one parameter of the yarn and further components. The front plate of the housing is used as a carrier element for the sensor device and several further components. In an especially preferred embodiment, the several further components are chosen from the set which contains the following elements: guide elements for yarn guidance, a sensor cover for the sensor device, an electronic circuit controlling parts of the apparatus and/or for evaluating the signals of the sensor device, a yarn conveying device, a roller cover for the yarn conveying device, a drive device for driving the yarn conveying device, a control of the drive device, a traversing device, a yarn suction device and a pressing cylinder for a yarn conveying roller. The sensor device and the several further components can be attached directly to the front plate and/or indirectly to angle sections, which on their part are attached to the front plate. This second embodiment also contributes to a simple and well-structured configuration of the apparatus and to its simple maintainability.

In a third embodiment, the apparatus in accordance with the invention for determining features of a yarn contains a sensor device for measuring at least one parameter of the yarn and a sensor cover for protecting the sensor device. The sensor cover comprises a measuring gap, through which the yarn is movable in its longitudinal direction, and an insertion slit through which the yarn is insertable into the measuring gap. The projection of the insertion slit into a plane perpendicular to the longitudinal direction is curved. In a preferred embodiment, the sensor device is an optoelectronic sensor device and the insertion slit is curved in such a way that light from the outside will not directly reach the yarn and/or a light-sensitive element of the sensor device, and/or that light emitted by a light-emitting element of the sensor device will not directly reach the exterior and/or via the yarn. As a result, the optoelectronic sensor device is shaded as well as possible from undesirable external light and the measuring signal is improved. On the other hand, work safety is improved because persons in the ambient environment of the apparatus cannot be injured by any escaping light. The term of “light” used in this specification relates not only to the visible range (VIS) of the electromagnetic spectrum, but also the adjacent ranges of ultraviolet (UV) and infrared (IR).

In a fourth embodiment, the apparatus in accordance with the invention for determining features of a yarn contains a sensor device for measuring at least one parameter of the yarn and a yarn conveying device for conveying the yarn through the sensor device. The yarn conveying device is arranged as a roller delivery mechanism with two cooperating conveying rollers. The axes of the conveying rollers are disposed in parallel with respect to each other and have a variable distance from one another, and at least one of the conveying rollers is driven. The apparatus further contains a limit sensor or a limit switch which interrupts the drive of the different conveying roller when the distance between the two axes exceeds a predetermined threshold value. This fourth embodiment therefore offers effective protection from undesirable ribbon formation and jamming of fingers, hair, and articles of clothing or jewelry.

In a fifth embodiment, the apparatus in accordance with the invention for determining features of a yarn contains a sensor device for measuring at least one parameter of the yarn and a yarn conveying device for conveying the yarn through the sensor device. The yarn conveying device is arranged as a roller delivery mechanism with two cooperating conveying rollers, with the axes of the conveying rollers being disposed in parallel with respect to each other. The apparatus further contains a traversing device which temporally changes the position of the yarn in the direction of the axes of the conveying rollers in the region of the yarn conveying device during yarn conveyance. For this purpose, the traversing device can contain a traversing element arranged as a yarn guide eye which is movable in a reciprocating fashion in the direction of the axes of the conveying rollers, e.g. by means of a pneumatic traversing cylinder. As a result of this fifth embodiment, the lateral surfaces of the conveying rollers are subjected to loads in an even fashion in the axial direction instead at only one position, thereby increasing their service life.

In a sixth embodiment, the apparatus in accordance with the invention for determining features of a yarn contains a sensor device for measuring at least one parameter of the yarn and a yarn conveying device for conveying the yarn through the sensor device. The yarn conveying device is arranged as a roller delivery mechanism with two cooperating conveying rollers, with at least one of the conveying rollers being drivable by a drive device. The at least one drivable conveying roller is drivable directly by the drive device. This provides the apparatus with a simple and cost-effective configuration. A flexible coupling can be attached between the drive device and the at least one drivable conveying roller, which coupling is arranged as a spiral coupling or a bellows coupling. This ensures even running properties of the yarn.

The aforementioned embodiments can be combined with one another at will. Such combinations lead to further especially advantageous embodiments.

The apparatus in accordance with the invention can be used for determining different features of a yarn. It can be suitable for determining only one single feature or several different features. An especially advantageous use of the apparatus in accordance with the invention is the optoelectronic measurement of the hairiness of the yarn.

### DRAWINGS

Various aspects of the invention will be explained in closer detail by reference to the drawings.

FIG. 1 shows an apparatus in accordance with the invention in a perspective view.

FIG. 2 shows the configuration of a housing of the apparatus in accordance with the invention.

FIG. 3 shows a front plate of the apparatus (a) in accordance with the invention in a front view and (b) in a rear view.

FIG. 4 shows a sensor cover of the apparatus in accordance with the invention in a perspective view.

FIG. 5 shows conveying rollers and a traversing device of the apparatus in accordance with the invention in perspective views (a) from above and (b) from the side.

FIG. 6 shows a coupling for the yarn drive of the apparatus in accordance with the invention in a side view.

### DESCRIPTION

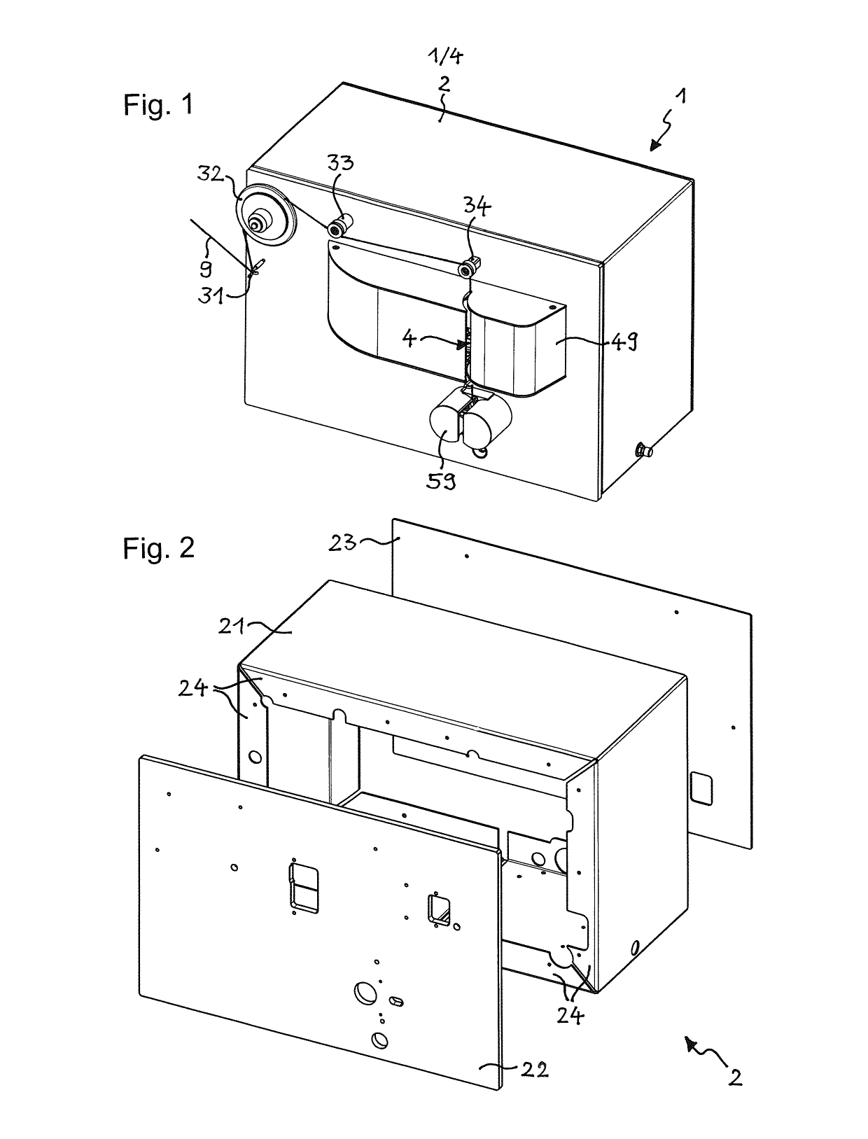

FIG. 1 shows an outside view of an apparatus 1 in accordance with the invention for determining features of a yarn 9. The illustration shows a substantially cuboid housing 2, various yarn guide elements 31 to 34 for guiding the yarn 9, a sensor cover 49 for a yarn sensor device 4, and a roller cover 59 for a yarn conveying device 5. The yarn sensor device 4 measures at least one parameter of the yarn 9, e.g. the length of fibers (“hairs”) which protrude from the yarn 9.

FIG. 2 shows an especially simple configuration of the housing 2 of the apparatus 1. The housing 2 substantially consists of a frame 21, a front plate 22 and a rear plate 23. The frame 21 is produced integrally, preferably in that a sheet metal is bent and welded together at the ends, so that substantially a lateral surface of a cuboid is obtained. Steel sheet of a thickness of approximately 2 mm for example can be used for the frame 21. The front plate 22 and the rear plate 23 are respectively arranged as flat metal plates. They can consist of steel or aluminum with a thickness of approximately 2 to 16 mm. The fastening of the front plate 22 and the rear plate 23 to the frame 21 can occur by means of screwed joints on lugs 24 which are bent inwardly on the frame 21.

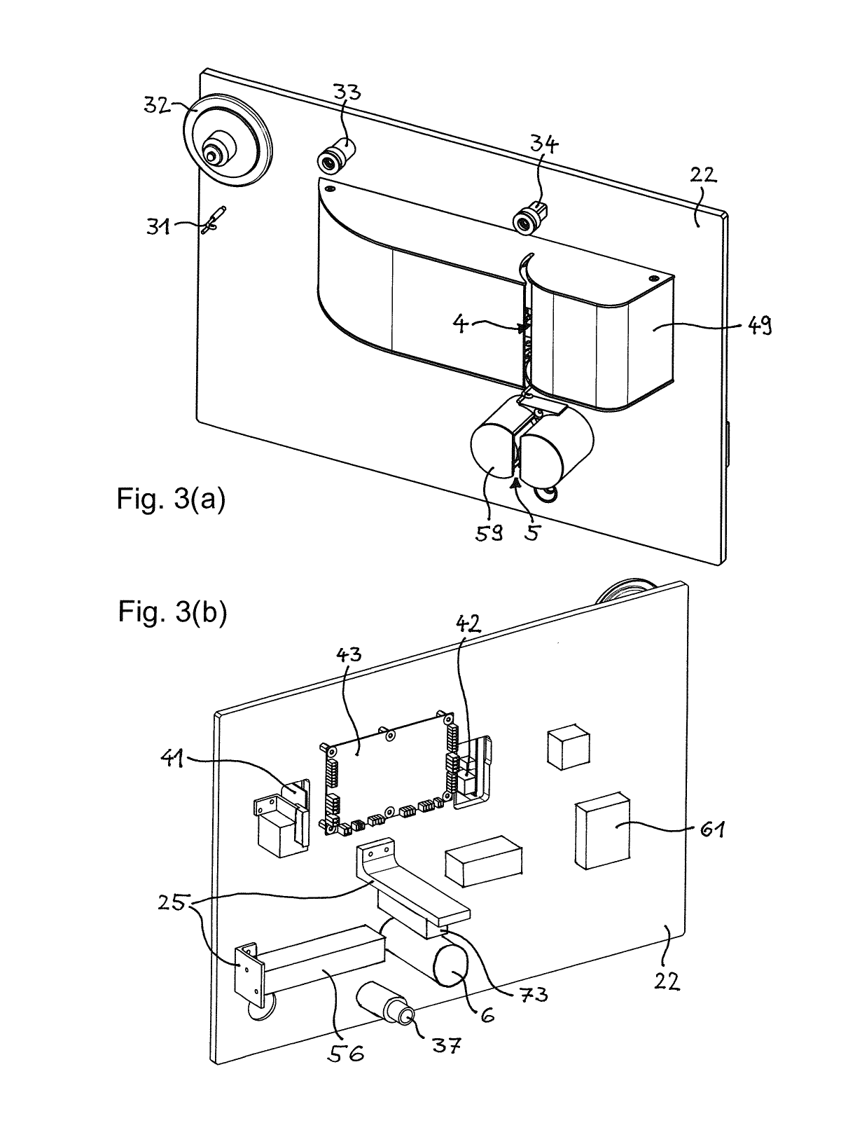

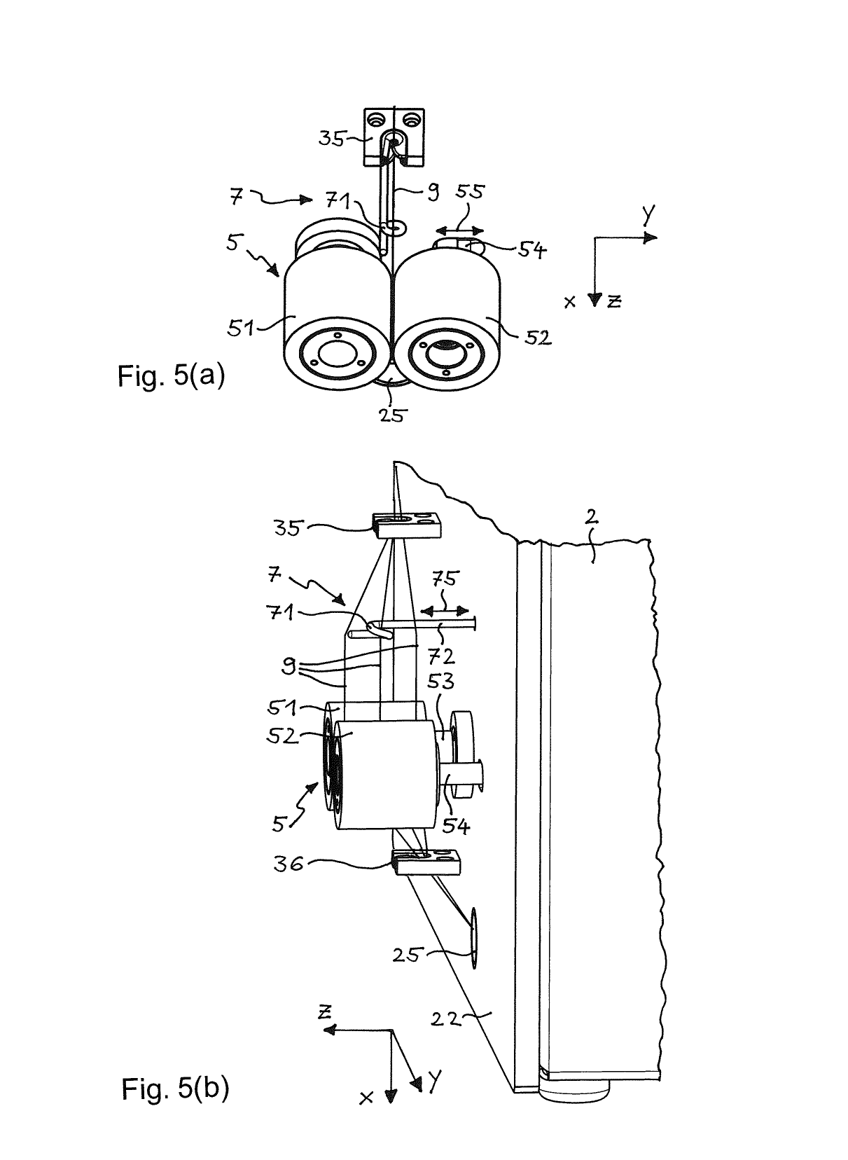

FIG. 3 shows the front plate 22 (a) in a front view and (b) in a rear view. The front plate 22 is used as the support element for as many components of the apparatus 1 as possible. In the example FIG. 3 the front plate 22 carries on its front side (FIG. 3(a)) the sensor cover 49, the sensor device 4 (which is covered by the sensor cover 49 and is partly shown in FIG. 3(b)) which can contain a light transmitter 41 and a light receiver 42 for example, the roller cover 59, the yarn conveying device 5 (which is covered by the roller cover 59), and the yarn guide elements 31 to 34. The rear side (FIG. 3(b)) of the front plate 22 comprises a printed circuit board 43 for example with an electronic circuit for controlling parts of the apparatus 1 and/or for evaluating the sensor signals, a drive control unit 61, a traversing cylinder 73, a drive device 6, a yarn suction device 37, and a pressure cylinder 56 for the pressure roller 52 (cf. FIG. 5). These and/or other components can be attached directly to the front plate 22 or indirectly to angle sections 25, which on their part are attached to the front plate 22.

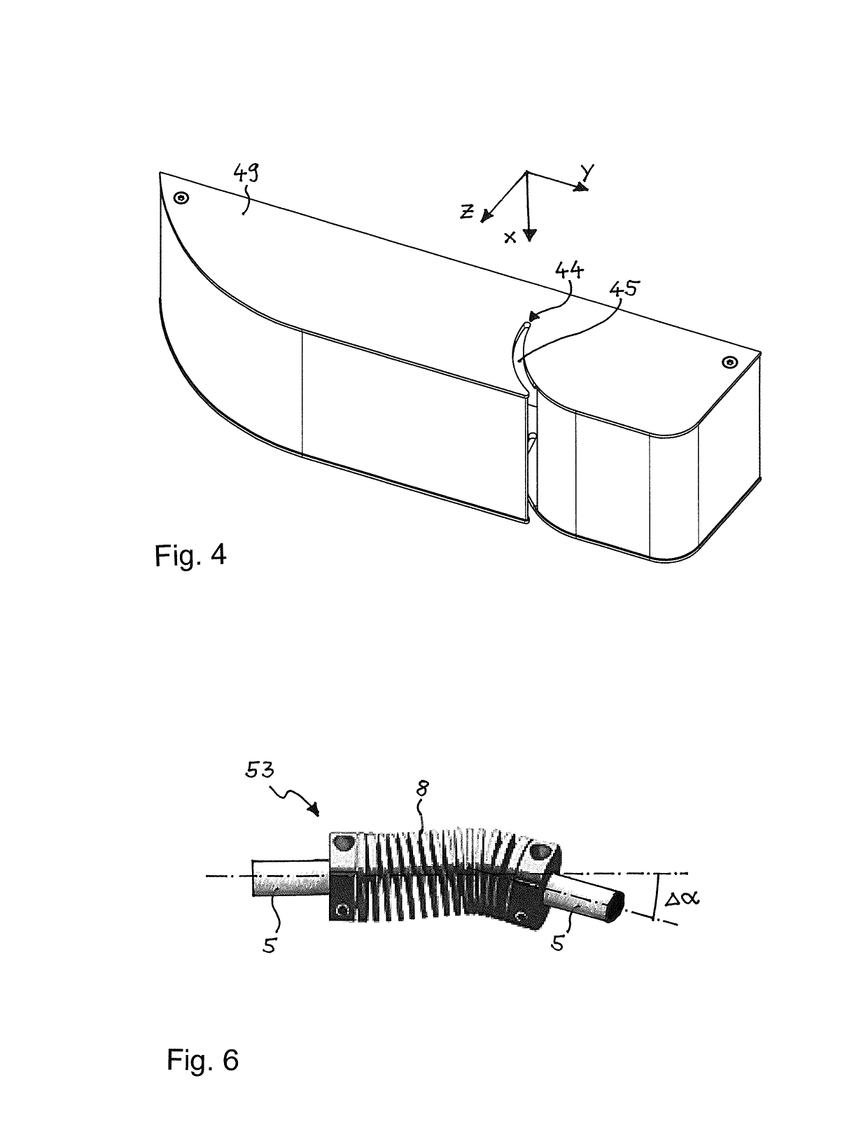

The sensor cover 49 of the apparatus 1 is shown in FIG. 4. The sensor cover 49 protects the sensor device 4 of the apparatus 1 (cf. FIG. 1) from mechanical effects, contamination and light from external sources in the case of an optoelectronic sensor device 4. In the latter case, the sensor cover 49 is non-transparent in the spectral range in which the optoelectronic sensor device 4 is light-sensitive. The sensor cover 49 comprises a measuring gap 44, through which the yarn 9 (not shown in FIG. 4) is moved in its longitudinal direction x, and an insertion slit 45 through which the yarn 9 can be inserted into the measuring gap 44. The measuring gap 44 and the insertion slit 45 extend parallel to the longitudinal direction x. In accordance with the invention, the projection of the insertion slit 45 into the plane (yz) perpendicularly to the longitudinal direction x is curved. Such a curvature offers a number of advantages. It prevents on the one hand that light from the outside impinges through the insertion slit 45 directly onto the yarn 9 and/or a light-sensitive element 42 (cf. FIG. 3(b)) of the sensor device 4. The optoelectronic sensor device 4 is consequently shaded as well as possible from undesirable external light, and the measuring signal is improved. On the other hand, the curvature also prevents that light emitted from the light transmitter 42 escapes to the outside directly and/or via the yarn 9 and potentially causes injuries to persons. Work safety is improved thereby.

FIG. 5 shows a yarn conveying device 5 and a traversing device 7 of the apparatus 1 in accordance with the invention (a) from above and (b) from the side. The yarn conveying device 5 is arranged as a roller delivery mechanism with two cooperating conveying rollers 51, 52. A first active drive roller 51 is driven for rotation. The drive can occur by a drive device 6 (see FIG. 3(b)) such as an electric motor via a shaft 53. A second passive pressure roller 52 is a non-driven roller which is rotatable about an axis 54. The lateral surfaces of the two conveying rollers 51, 52 are suitable for making contact and entraining the yarn 9 to be conveyed and consist of metal or an elastomeric material for example. The shaft 53 and the axis 54 of the two conveying rollers 51 and 52 are parallel with respect to each other. The two lateral surfaces touch one another when no yarn 9 has been inserted. Otherwise, they will clamp the yarn 9 between themselves and will convey it in the longitudinal direction x through the sensor device 4 (not shown in FIG. 5).

The axis 54 of the pressure roller 52 is movable in the direction y perpendicular to the axial direction z and perpendicular to the longitudinal direction x of the yarn within a small range of 3 mm for example, which is indicated in FIG. 5(a) by a double arrow 55. It is subjected to a force which presses the pressure roller 52 against the drive roller 51, e.g. by means of a pneumatic pressure cylinder 56 (see FIG. 3(b)). When the axis 54 reaches a predetermined outer position, a limit sensor or a limit switch (not shown) automatically deactivates the drive device 6. It is thereby ensured on the one hand that the drive device 6 will not run in the open position of the conveying rollers 51, 52, e.g. when the yarn 9 is inserted. On the other hand, the formation of a ribbon is thereby prevented. A ribbon means that the yarn 9 starts to wind up in an undesirable way on one of the conveying rollers 51, 52. If this is the case, the wound yarn 9 increases the distance between the shaft 53 and axis 54, so that the limit switch will finally respond and deactivate the drive device 6. Similarly, the drive device will be shut off when fingers, hair, items of clothing or jewelry come to be between the conveying rollers 51, 52. Thirdly, accidents and injuries are therefore avoided.

A further aspect of the invention relates to yarn traversing. This is understood to be a temporal change in the position of the yarn in the direction z of the shaft 53 and the axis 54 in order to evenly load the lateral surfaces of the conveying rollers 51, 52 in the axial direction z instead of only one position. Yarn traversing is achieved in accordance with the invention in such a way that in the region of the conveying device 5 the position of the yarn 9 is changed in the axial direction z by means of a traversing device 7 containing a traversing element 71. The traversing movement is indicated with a double arrow 75 in FIG. 5(b), and the yarn 9 is shown in three different positions. In the embodiment as discussed here, the traversing element 71 is arranged as a yarn guide eye which is attached to the end of a rod 72 which is movable in the axial direction z. The rod 72 and therefore the yarn guide eye 71 is slowly moved back and forth in the axial direction z by means of a pneumatic traversing cylinder 73 (see FIG. 3(b)). “Slow” shall mean in this context that several meters of the yarn are conveyed by the conveying device 5 during a traversing cycle, e.g. 2 to 20 m, and preferably 6 to 12 m. The traversing device 7 is disposed upstream with respect to the conveying rollers 51, 52. A first stationary yarn guide element 35 is attached between the traversing device 7 and the sensor device 4 and ensures stable stationary run of the yarn through the sensor device. Preferably, a second stationary yarn guide element 36 is attached downstream with respect to the conveying rollers 51, 52. Further stationary yarn guide elements can also be provided in the region of the sensor device 4, e.g. on the sensor cover 49. At least one of the stationary yarn guide elements 35, 36 can be arranged as a trap eyelet, in which the insertion of the yarn 9 from the front, i.e. through the insertion slit 45, is simple, but extraction in the opposite direction is difficult. The yarn 9 which has been conveyed through the sensor device 4 is sucked off by means of the yarn suction device 37 (see FIG. 3(b)) through a suction opening 25 in the front plate 22 to a disposal container (not shown).

The drive roller 51 is driven directly by the drive device 6, i.e. without the otherwise usual transmission belts or gears. This provides the apparatus 1 with an especially simple and cost-effective configuration. It is advantageous to provide a flexible coupling 8 on the shaft 53 between the drive device 6 and the drive roller 51, which coupling compensates a possible local offset and/or angular offset between the drive shaft 57 and the output shaft 58, and thereby ensures even run of the yarn. Such a flexible coupling 8 is shown in FIG. 6. Preferably, a spiral coupling or a bellows coupling made of metal or plastic is used.

It is understood that the present invention is not limited to the embodiment as discussed above. The person skilled in the art will be able to derive further variants with knowledge of the invention which also belong to the subject matter of the present invention as defined in the independent claims.

### REFERENCE NUMERALS

1 Apparatus
2 Housing
21 Frame
22 Front plate
23 Rear plate
24 Lugs
25 Suction opening
31-34 Yarn guide elements
35, 36 Stationary yarn guide elements
4 Sensor device
41 Light transmitter
42 Light receiver
43 Printed circuit board
44 Measuring gap
45 Insertion slit
49 Sensor cover
5 Yarn conveying device
51 Drive roller
52 Pressure roller
53 Shaft of the drive roller
54 Axis of the pressure roller
55 Movement of the axis
56 Pressure cylinder for the pressure roller
57 Drive shaft
58 Output shaft
59 Roller cover
6 Drive device
7 Traversing device
71 Traversing element
72 Rod
73 Traversing cylinder
75 Traversing movement
8 Flexible coupling
9 Yarn
x Longitudinal direction of the yarn, running direction of the yarn
y Direction perpendicular to the longitudinal direction of the yarn, parallel to the front plate
z Direction perpendicular to the longitudinal direction of the yarn, perpendicular to the front plate

1. An apparatus for determining the features of a yarn, comprising:
a housing, and
a sensor device attached in or to the housing for measuring at least one parameter of the yarn,
wherein,
the housing includes a front plate, a rear plate and a frame disposed between the front plate and the rear plate, and
the frame is produced integrally and separately from the front plate and the rear plate and is made of a bent sheet metal.

2. The apparatus according to claim 1, wherein the front plate and the rear plate are arranged as flat plates and are fastened by screw connections to lugs that are inwardly bent off on the frame.

3. The apparatus according to claim 1, wherein the front plate stands substantially perpendicularly on a base, and the front plate is used as a support element for the sensor device and other components.

4. The apparatus according to claim 3, wherein the other components are selected from the set consisting of: guide elements for yarn guidance, a sensor cover for the sensor device, an electronic circuit for at least one of controlling parts of the apparatus and evaluating signals of the sensor device, a yarn conveying device, a roller cover for the yarn conveying device, a drive device for driving the yarn conveying device, a control unit for the drive device, a traversing device, a yarn suction device, and a pressure cylinder for a yarn conveying roller.

5. The apparatus according to claim 3, wherein the sensor device and the other components are directly attached to the front plate or indirectly to angle sections that are fastened to the front plate.

6. The apparatus according to claim 1, further comprising a yarn conveying device for conveying the yarn through the sensor device, the yarn conveying device including a roller delivery mechanism with two cooperating conveying rollers, wherein at least one of the conveying rollers is driven by a drive device, and a flexible coupling is attached between the drive device and the at least one drivable conveying roller, wherein the coupling is one of a spiral coupling and a bellows coupling.

7. The apparatus according to claim 1, further comprising a sensor cover for protection of the sensor device, wherein the sensor cover comprises a measuring gap through which the yarn is movable in its longitudinal direction, and an insertion slit through which the yarn is insertable into the measuring gap, and wherein the projection of the insertion slit into a plane perpendicular to the longitudinal direction is curved.

8. The apparatus according to claim 7, wherein the sensor device is an optoelectronic sensor device and the insertion slit is curved in such a way that at least one of (a) light from the outside will not directly reach at least one of the yarn, a light-sensitive element of the sensor device, and (b) that light emitted by a light-emitting element of the sensor device will not reach the outside at least one of directly and via the yarn.

9. The apparatus according to claim 1, further comprising a yarn conveying device for conveying the yarn through the sensor device, the yarn conveying device including a roller delivery mechanism with two cooperating conveying rollers, wherein axes of the conveying rollers are disposed in parallel with respect to each other and have a variable distance from each other, and at least one of the conveying rollers is driven, and wherein a limit sensor or limit switch interrupts the drive of the driven conveying roller when the distance between the two axes has exceeded a predetermined threshold value.

10. The apparatus according to claim 1, further comprising a yarn conveying device for conveying the yarn through the sensor device, the yarn conveying device including a roller delivery mechanism with two cooperating conveying rollers, with the axes of the conveying rollers being disposed in parallel with respect to each other, wherein a traversing device temporally changes the position of the yarn in the direction of the axes of the conveying rollers in the region of the yarn conveying device during the yarn conveyance.

11. The apparatus according to claim 10, wherein the traversing device includes a traversing element having a yarn guide eye that is movable back and forth in the direction of the axes of the conveying rollers.

12. The apparatus according to claim 11, wherein the traversing element is movable back and forth by a pneumatic traversing cylinder.

13. An apparatus for determining the features of a yarn, comprising:
a sensor device for measuring at least one parameter of the yarn, and
a sensor cover for protecting the sensor device, wherein, the sensor cover comprises,
a measuring gap through which the yarn is movable in its longitudinal direction, and
an insertion slit through which the yarn is insertable into the measuring gap,
wherein the projection of the insertion slit into a plane perpendicular to the longitudinal direction is curved.
